(19) United States
(12) Patent Application Publication
(10) Pub. No.: US 2026/0345021 A1
(43) Pub. Date: Nov. 5, 2026

(54) LASER DIFFUSION-BASED MOTION ROBUST HEART RATE MONITORING

(71) Applicant: Samsung Electronics Co., Ltd., Suwon-si (KR)

(72) Inventors: Guanbo Chen, Hillsborough, NJ (US); Indranil Sinharoy, Richardson, TX (US); Gang Xu, Allen, TX (US); Yan Xin, Princeton, NJ (US); Jianzhong Zhang, Dallas, TX (US)

(21) Appl. No.: 19/655,517

(22) Filed: Apr. 22, 2026

Related U.S. Application Data
(60) Provisional application No. 63/797,736, filed on Apr. 30, 2025.

(51) Int. Cl. A61B 5/024; A61B 5/00; A61B 5/026

(52) U.S. Cl. CPC A61B 5/02427; A61B 5/0261; A61B 5/721; A61B 5/7264; A61B 5/681

(57) ABSTRACT

Apparatuses and methods for laser diffusion-based motion robust heart rate monitoring. A method for laser sensor system for direct heart rate signal measurement without interference from human body motion includes detecting one or more blood flow signals under human skin for estimation of a heart rate, using a spatially offset pair of laser transmitter and laser receiver. The method includes detecting one or more human motion signals, using a spatially adjacent pair of laser transmitter and laser receiver. The method includes reducing a motion interference signal within the one or more blood flow signals by filtering the one or more human motion signals out from the one or more blood flow signals. The method includes monitoring the heart rate based on the one or more filtered blood flow signals.

### CROSS-REFERENCE TO RELATED APPLICATION AND CLAIM OF PRIORITY

This application claims priority under 35 U.S.C. § 119 (e) to U.S. Provisional Patent Application No. 63/797,736 filed on Apr. 30, 2025. The above-identified provisional patent application is hereby incorporated by reference in its entirety.

### TECHNICAL FIELD

This disclosure relates generally to heart rate monitoring. More specifically, this disclosure relates to laser diffusion-based motion robust heart rate monitoring.

### BACKGROUND

Recent electronic devices come in various form factors for user convenience purposes and in reduced size for easy carrying. For example, the electronic device may be provided in the form of a wearable fitness wristband or smartwatch worn on the user's wrist. As another example, the electronic device may be a ring worn on the user's finger. More interest is being paid to health, and so is technology capable of health condition monitoring, such as heart heat monitoring. Accordingly, electronic devices include a sensor for measuring the user's biometric information and utilize various biometric signals sensed by the sensor to provide various services for the user's health care or health condition monitoring through measurement of various biometric signals.

### SUMMARY

This disclosure provides laser diffusion-based motion robust heart rate monitoring.

In one embodiment, a method for laser sensor system for direct heart rate signal measurement without interference from human body motion is provided. The method includes detecting one or more blood flow signals under human skin for estimation of a heart rate, using a spatially offset pair of laser transmitter and laser receiver. The method includes detecting one or more human motion signals, using a spatially adjacent pair of laser transmitter and laser receiver. The method includes reducing a motion interference signal within the one or more blood flow signals by filtering the one or more human motion signals out from the one or more blood flow signals. The method includes monitoring the heart rate based on the one or more filtered blood flow signals.

In another embodiment, an electronic device for laser sensor system for direct heart rate signal measurement without interference from human body motion is provided. The electronic device includes a spatially offset pair of laser transmitter and laser receiver, a spatially adjacent pair of laser transmitter and laser receiver, and a processor operably connected to each laser transmitter and each laser receiver from among the spatially offset pair and the spatially adjacent pair. The processor is configured to detect one or more blood flow signals under human skin for estimation of a heart rate, using a spatially offset pair of laser transmitter and laser receiver. The processor is configured to detect one or more human motion signals, using a spatially adjacent pair of laser transmitter and laser receiver. The processor is configured to reduce a motion interference signal within the one or more blood flow signals by filtering the one or more human motion signals out from the one or more blood flow signals. The processor is configured to monitor the heart rate based on the one or more filtered blood flow signals.

In yet another embodiment, a non-transitory computer readable medium comprising program code for laser sensor system for direct heart rate signal measurement without interference from human body motion is provided. The computer program includes computer readable program code that when executed causes at least one processor to detect one or more blood flow signals under human skin for estimation of a heart rate, using a spatially offset pair of laser transmitter and laser receiver. The computer readable program code causes the processor to detect one or more human motion signals, using a spatially adjacent pair of laser transmitter and laser receiver. The computer readable program code causes the processor to reduce a motion interference signal within the one or more blood flow signals by filtering the one or more human motion signals out from the one or more blood flow signals. The computer readable program code causes the processor to monitor the heart rate based on the one or more filtered blood flow signals.

Other technical features may be readily apparent to one skilled in the art from the following figures, descriptions, and claims.

Before undertaking the DETAILED DESCRIPTION below, it may be advantageous to set forth definitions of certain words and phrases used throughout this patent document. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

As used here, terms and phrases such as “have,” “may have,” “include,” or “may include” a feature (like a number, function, operation, or component such as a part) indicate the existence of the feature and do not exclude the existence of other features. Also, as used here, the phrases “A or B,” “at least one of A and/or B,” or “one or more of A and/or B” may include all possible combinations of A and B. For example, “A or B,” “at least one of A and B,” and “at least one of A or B” may indicate all of (1) including at least one A, (2) including at least one B, or (3) including at least one A and at least one B. Further, as used here, the terms “first” and “second” may modify various components regardless of importance and do not limit the components. These terms are only used to distinguish one component from another. For example, a first user device and a second user device may indicate different user devices from each other, regardless of the order or importance of the devices. A first component may be denoted a second component and vice versa without departing from the scope of this disclosure.

It will be understood that, when an element (such as a first element) is referred to as being (operatively or communicatively) “coupled with/to” or “connected with/to” another element (such as a second element), it can be coupled or connected with/to the other element directly or via a third element. In contrast, it will be understood that, when an element (such as a first element) is referred to as being “directly coupled with/to” or “directly connected with/to” another element (such as a second element), no other element (such as a third element) intervenes between the element and the other element.

As used here, the phrase “configured (or set) to” may be interchangeably used with the phrases “suitable for,” “having the capacity to,” “designed to,” “adapted to,” “made to,” or “capable of” depending on the circumstances. The phrase “configured (or set) to” does not essentially mean “specifically designed in hardware to.” Rather, the phrase “configured to” may mean that a device can perform an operation together with another device or parts. For example, the phrase “processor configured (or set) to perform A, B, and C” may mean a generic-purpose processor (such as a CPU or application processor) that may perform the operations by executing one or more software programs stored in a memory device or a dedicated processor (such as an embedded processor) for performing the operations.

The terms and phrases as used here are provided merely to describe some embodiments of this disclosure but not to limit the scope of other embodiments of this disclosure. It is to be understood that the singular forms “a,” “an,” and “the” include plural references unless the context clearly dictates otherwise. All terms and phrases, including technical and scientific terms and phrases, used here have the same meanings as commonly understood by one of ordinary skill in the art to which the embodiments of this disclosure belong. It will be further understood that terms and phrases, such as those defined in commonly-used dictionaries, should be interpreted as having a meaning that is consistent with their meaning in the context of the relevant art and will not be interpreted in an idealized or overly formal sense unless expressly so defined here. In some cases, the terms and phrases defined here may be interpreted to exclude embodiments of this disclosure.

Definitions for other certain words and phrases may be provided throughout this patent document. Those of ordinary skill in the art should understand that in many if not most instances, such definitions apply to prior as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 illustrates an example communication system in accordance with an embodiment of this disclosure;

FIGS. 2 and 3 illustrate example electronic devices a communication system in accordance with an embodiment of this disclosure;

FIGS. 4A and 4B illustrate a side view and a top view of an example system diagram of a laser diffusion-based motion robust heart rate monitoring sensor in accordance with an embodiment of this disclosure;

FIG. 4C illustrates a bottom perspective view of a smartwatch that includes hardware architecture of the laser diffusion-based motion robust heart rate monitoring sensor in accordance with an embodiment of this disclosure;

FIG. 5A illustrates an example usual scenario of reflected signals from the skin surface exhibiting difficulty or inability to reach a spatially offset laser receiver in accordance with an embodiment of this disclosure;

FIG. 5B illustrates an example rare scenario of reflected signals from the skin surface that reach a spatially offset laser receiver in accordance with an embodiment of this disclosure;

FIG. 6 illustrates a non-laser diffusion-based motion robust heart rate monitoring sensor that includes a light emitting diode (LED) light source;

FIG. 7 illustrates an example sequential signal processing (SSP) model in accordance with an embodiment of this disclosure;

FIG. 8 illustrates an example signal processing operation of the SSP model of FIG. 7 for robust heart rate estimation in accordance with an embodiment of this disclosure;

FIG. 9 illustrates an example a wearable device including an integrated optical sensor (IOS) for measuring human vital signs in accordance with an embodiment of this disclosure;

FIG. 10 illustrates an example a wearable device including a second hardware architecture of an integrated optical sensor (IOS) for measuring human vital signs in accordance with an embodiment of this disclosure;

FIG. 11 illustrates an example a wearable device including a third hardware architecture of an IOS for measuring human vital signs in accordance with an embodiment of this disclosure;

FIG. 12 illustrates an example a wearable device including a fourth hardware architecture of a time-domain diffusion correlation spectroscopy (TD-DCS) based robust heart rate monitoring sensor in accordance with an embodiment of this disclosure;

FIG. 13 illustrates an example process for generating a motion artefact-free SPG signal from two two-dimensional detectors in accordance with an embodiment of this disclosure; and

FIG. 14 illustrates a method for laser sensor system for direct heart rate signal measurement without interference from human body motion in accordance with an embodiment of this disclosure.

### DETAILED DESCRIPTION

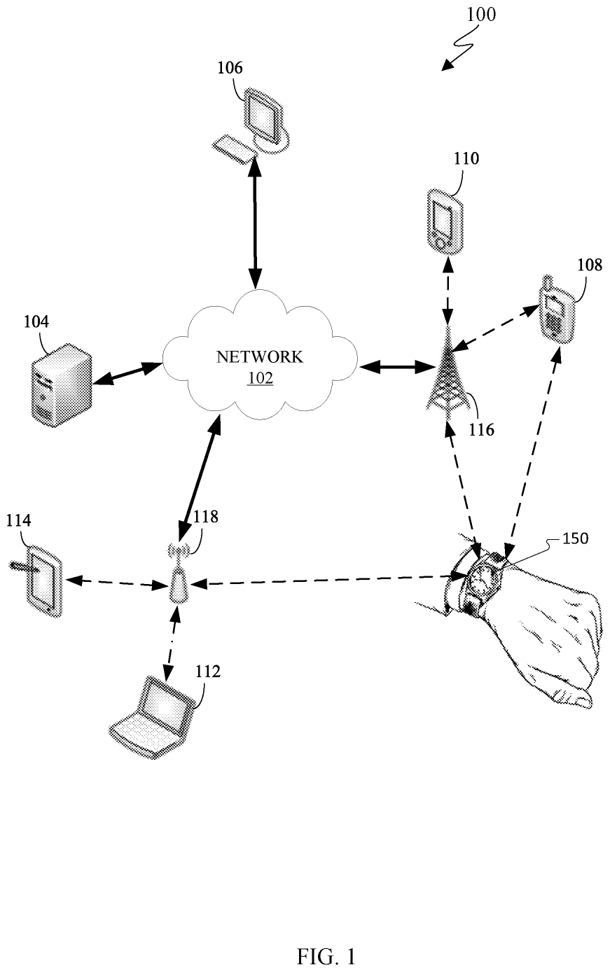

FIGS. 1 through 14, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably-arranged wireless communication system or device.

A motion robust heart rate sensor is a specialized device designed to accurately measure heart rate even during significant movement, overcoming the major challenge of heart rate sensors that are easily disrupted by body motions, like walking, running, or even simple arm gestures. This technology dominates in applications where continuous, reliable heart rate monitoring is needed while individuals are engaged in dynamic activities, including fitness tracking, medical monitoring for patients with mobility issues, and remote health monitoring systems where precise data acquisition during movement is advantageous.

One wearable heart rate monitoring sensor is a photoplethysmogram (PPG), which obtains plethysmogram optically, and the plethysmogram is used to estimate heart rate. PPG is an optic absorption based method in which the signal is affected by the volumetric change of the blood in the microvascular under skin as well as the optic signals scattered from the surface of the skin. Human body motion will have a significant impact to the optical signal path of the PPG sensor which will cause errors of the estimated heart rate. To address this problem, various methods have been proposed, including methods that use the measurement data from an accelerometer to approximate the motion signal. Then through adaptive filtering, the accelerometer-based motion signal is cancelled out from the measured PPG signal. However, as a disadvantage, this method may need the electronic device to include an additional accelerometer sensor which increases the hardware cost, and this accelerometer-based method might fail during very large movements. To overcome the problems of this accelerometer-based method, this disclosure provides an optical sensor based technique that can measure the heart rate while rejecting the interference from large motion.

Optical based heart rate monitoring is susceptible to body motions. The estimated heart rate can have approximately 5~15 beats per minute (bpm) errors due to large body motions. This disclosure provides an optical sensor that can accurately measure the heart rate while rejecting the motion signals. In addition, this disclosure further enhances the heart rate estimation results, by providing methods to detect human body motion signals and then cancel the human body motion signals out from the overall heart rate measurement signals.

There are multiple problems that embodiments of this disclosure solve. To solve one problem, this disclosure provides a sensor system developed to measure while rejecting the motion interference signal. Another problem is a need to develop a sensor system which can directly measure the motion signal. This disclosure solves yet another problem by providing a method that cancels out the motion signal from the heartbeat signal. For simplicity, the term “heartbeat signal” is interchangeable with “heart rate signal” in this disclosure, but it is understood that the heart rate can be extracted from the heartbeat signal.

FIG. 1 illustrates an example communication system 100 according to this disclosure. The embodiment of the communication system 100 shown in FIG. 1 is for illustration only. Other embodiments of the communication system 100 could be used without departing from the scope of this disclosure.

As shown in FIG. 1, the system 100 includes a network 102, which facilitates communication between various components in the system 100. For example, the network 102 may communicate Internet Protocol (IP) packets, frame relay frames, Asynchronous Transfer Mode (ATM) cells, or other information between network addresses. The network 102 may include one or more local area networks (LANs), metropolitan area networks (MANs), wide area networks (WANs), all or a portion of a global network such as the Internet, or any other communication system or systems at one or more locations.

The network 102 facilitates communications between at least one server 104 and various client devices 106, 108, 110, 112, 114, and 150. Each server 104 includes any suitable computing or processing device that can provide computing services for one or more client devices. Each server 104 could, for example, include one or more processing devices, one or more memories storing instructions and data, and one or more network interfaces facilitating communication over the network 102.

Each client device 106, 108, 110, 112, 114, and 150 represents any suitable computing or processing device that interacts with at least one server or other computing device(s) over the network 102. In this example, the client devices 106, 108, 110, 112, 114, and 150 include a desktop computer 106, a mobile telephone or smartphone 108, a personal digital assistant (PDA) 110, a laptop computer 112, a tablet computer 114, and a wearable electronic device 150. However, any other or additional client devices could be used in the computing system 100.

In this example, some client devices 106, 108, 110, 112, 114, and 150 communicate indirectly with the network 102. For example, the client devices 108, 110, and 150 communicate via one or more base stations 116, such as cellular base stations or eNodeBs or gNodeBs. Also, the client devices 112, 114, and 150 communicate via one or more wireless access points 118, such as IEEE 802.11 wireless access points. Note that these are for illustration only and that each client device could communicate directly with the network 102 or indirectly with the network 102 via any suitable intermediate device(s) or network(s).

The communication system 100 includes the base station 116 (e.g., BS, gNB). The base station 116 also communicates with at least one network 102, such as the Internet, a proprietary Internet Protocol (IP) network, or other data network. The base station 116 provides wireless broadband access to the network 102 for the client devices 108, 110, 150 within a coverage area of the base station 116.

Depending on the network type, the term “base station” or “BS” can refer to any component (or collection of components) configured to provide wireless access to a network, such as transmit point (TP), transmit-receive point (TRP), an enhanced base station (eNodeB or eNB), a 5G/NR base station (gNB), a macrocell, a femtocell, a WiFi access point (AP), or other wirelessly enabled devices. Base stations may provide wireless access in accordance with one or more wireless communication protocols, e.g., 5G/NR 3rd generation partnership project (3GPP) NR, long term evolution (LTE), LTE advanced (LTE-A), high speed packet access (HSPA), Wi-Fi 802.11a/b/g/n/ac, etc. For the sake of convenience, the terms “BS” and “TRP” are used interchangeably in this patent document to refer to network infrastructure components that provide wireless access to remote terminals. Also, depending on the network type, the term “client device” can refer to any component such as “user equipment” or “UE,” “mobile station,” “subscriber station,” “remote terminal,” “wireless terminal,” “receive point,” or “user device.” For the sake of convenience, the terms “user equipment” and “UE” are used in this patent document to refer to remote wireless equipment that wirelessly accesses a BS, whether the UE is a mobile device (such as a mobile telephone or smartphone) or is normally considered a stationary device (such as a desktop computer or vending machine).

The communication system 100 includes the access point 118 (e.g., AP). The AP 118 also communicates with at least one network 102, such as the Internet, a proprietary Internet Protocol (IP) network, or other data network. The AP 118 provides wireless access to the network 102 for the client devices 112, 114, 150 within a coverage area of the AP 118. The AP 118 can be a WiFi access point (AP) that provides wireless access in accordance with one or more wireless communication protocols, such as Wi-Fi 802.11a/b/g/n/ac, etc.

As described in more detail below, the client devices including the wearable electronic device 150 are used to measure bodily parameters such as heart rate and the like. The client device 150 can include a wristwatch, armband, torso-band, one or more patches, or the like. For example, the wearable electronic device 150 can communicate directly with another client device (such as client device 108) or can communicate indirectly with other client devices and the server 104 via a base station 116, an access point 118, or a client device (such as client device 108). As another example, the wearable electronic device 150 can output bodily parameters via an interface on the wearable electronic device 150 or via an interface on another client device (such as client device 108). The wearable electronic device 150 can also transmit bodily parameters to the server 104 for data storage and to be accessed by an authorized party.

Although FIG. 1 illustrates one example of a computing system 100, various changes may be made to FIG. 1. For example, the system 100 could include any number of each component in any suitable arrangement. In general, computing and communication systems come in a wide variety of configurations, and FIG. 1 does not limit the scope of this disclosure to any particular configuration. While FIG. 1 illustrates one operational environment in which various features disclosed in this patent document can be used, these features could be used in any other suitable system.

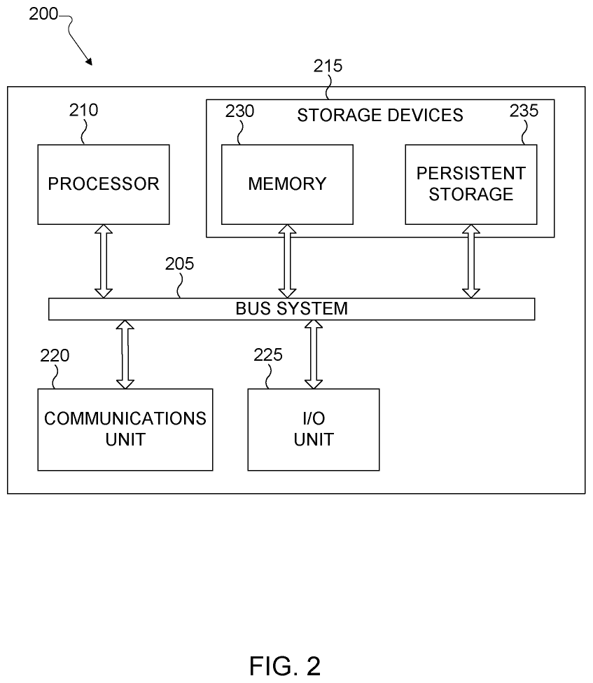

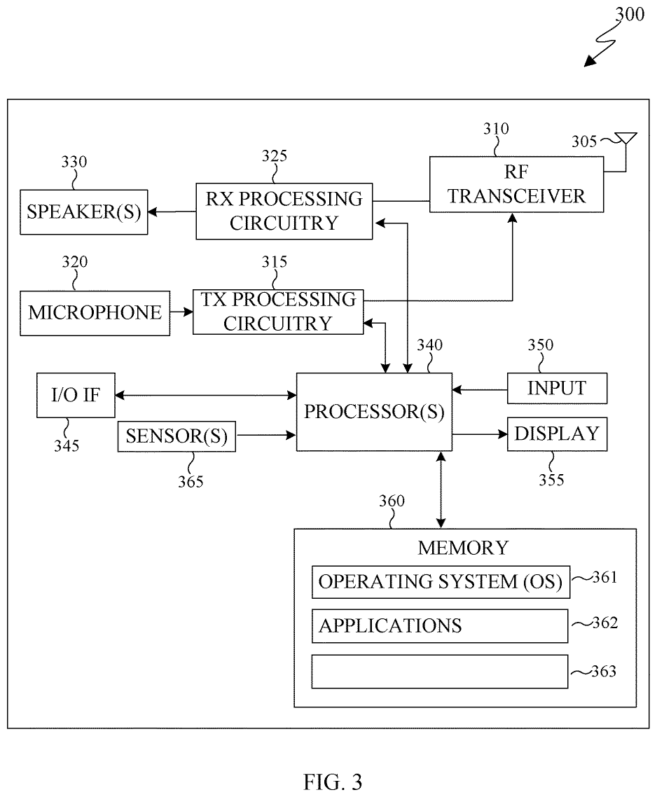

FIGS. 2 and 3 illustrate example devices in a communication system according to this disclosure. In particular, FIG. 2 illustrates an example server 200, and FIG. 3 illustrates an example client device 300. The server 200 could represent the server 104 in FIG. 1, and the client device 300 could represent one or more of the client devices 106, 108, 110, 112, 114, or 150 in FIG. 1.

As shown in FIG. 2, the server 200 includes a bus system 205, which supports communication between at least one processor 210, at least one storage device 215, at least one communications unit 220, and at least one input/output (I/O) unit 225.

The processor 210 executes instructions that may be loaded into a memory 230. The processor 210 may include any suitable number(s) and type(s) of processors or other devices in any suitable arrangement. Example types of processors 210 include microprocessors, microcontrollers, digital signal processors, field programmable gate arrays, application specific integrated circuits, and discreet circuitry.

The memory 230 and a persistent storage 235 are examples of storage devices 215, which represent any structure(s) capable of storing and facilitating retrieval of information (such as data, program code, and/or other suitable information on a temporary or permanent basis). The memory 230 may represent a random access memory or any other suitable volatile or non-volatile storage device(s). The persistent storage 235 may contain one or more components or devices supporting longer-term storage of data, such as a ROM, hard drive, Flash memory, or optical disc.

The communications unit 220 supports communications with other systems or devices. For example, the communications unit 220 could include a network interface card or a wireless transceiver facilitating communications over the network 102. The communications unit 220 may support communications through any suitable physical or wireless communication link(s).

The I/O unit 225 allows for input and output of data. For example, the I/O unit 225 may provide a connection for user input through a keyboard, mouse, keypad, touchscreen, or other suitable input device. The I/O unit 225 may also send output to a display, printer, or other suitable output device.

Note that while FIG. 2 is described as representing the server 104 of FIG. 1, the same or similar structure could be used in one or more of the client devices 106, 108, 110, 112, 114, and 150. For example, a laptop or desktop computer could have the same or similar structure as that shown in FIG. 2.

As shown in FIG. 3, the client device 300 includes an antenna 305, a radio frequency (RF) receiver 310, an RF transmitter 312, transmit (TX) processing circuitry 315, a microphone 320, and receive (RX) processing circuitry 325. The client device 300 also includes a speaker 330, a processor 340, an input/output (I/O) interface (IF) 345, an input 350, a display 355, and a memory 360. The memory 360 includes an operating system (OS) program 361 and one or more applications 362. The client device 300 also includes one or more sensor(s) 365.

The RF receiver 310 receives, from the antenna 305, an incoming RF signal transmitted by another component in a system. The RF receiver 310 down-converts the incoming RF signal to generate an intermediate frequency or baseband signal. The intermediate frequency or baseband signal is sent to the RX processing circuitry 325, which generates a processed baseband signal by filtering, decoding, and/or digitizing the baseband or IF signal. The RX processing circuitry 325 transmits the processed baseband signal to the speaker 330 (such as for voice data) or to the processor 340 for further processing (such as for web browsing data or for calculating bodily parameters based on the received RF signal).

The TX processing circuitry 315 receives analog or digital voice data from the microphone 320 or other outgoing baseband data (such as web data, e-mail, or interactive video game data, or a command to measure a bodily parameter) from the processor 340. The TX processing circuitry 315 encodes, multiplexes, and/or digitizes the outgoing baseband data to generate a processed baseband or intermediate frequency signal. The RF transmitter 312 receives the outgoing processed baseband or intermediate frequency signal from the TX processing circuitry 315 and up-converts the baseband or intermediate frequency signal to an RF signal that is transmitted via the antenna 306. In an embodiment, the two or more network access interfaces can include one or more I/O IFs 345, one or more RF receivers 310, one or more RF transmitters 312, or the like. The I/O IF 345 can communicate via a wired connection such as a network interface card for an Ethernet connection or a cable interface for a set top box. The RF receiver 310 can communicate with a wireless access point (such as wireless access point 118), a base station (such as base station 116), the RF transmitter 312, or the like. The RF transmitter 312 can communicate with a wireless access point (such as wireless access point 118), a base station (such as base station 116), the RF receiver 310, or the like.

The processor 340 can include one or more processors or other processing devices and execute the OS program 361 stored in the memory 360 in order to control the overall operation of the client device 300. For example, the processor 340 could control the reception of forward channel signals and the transmission of reverse channel signals by the RF receiver 310, the RF receiver, the RX processing circuitry 325, and the TX processing circuitry 315 in accordance with well-known principles. In some embodiments, the processor 340 includes at least one microprocessor or microcontroller.

The processor 340 is also capable of executing other processes and programs resident in the memory 360. The processor 340 can move data into or out of the memory 360 as required by an executing process. In some embodiments, the processor 340 is configured to execute the applications 362 based on the OS program 361 or in response to signals received from external devices or an operator. The processor 340 is also coupled to the I/O interface 345, which provides the client device 300 with the ability to connect to other devices such as laptop computers and handheld computers. The I/O interface 345 is the communication path between these accessories and the processor 340.

The processor 340 is also coupled to the input 350 and the display unit 355. The operator of the client device 300 can use the input 350 to enter data into the client device 300. The display 355 may be a liquid crystal display or other display capable of rendering text and/or at least limited graphics, such as from web sites.

The memory 360 is coupled to the processor 340. Part of the memory 360 could include RAM, and another part of the memory 360 could include a Flash memory or other ROM.

As described in more detail below, the client device 300 is configured to emit (for example, transmit) a laser or optical signal to a target such a region of a body of a living being such as a region of a human body. The client device 300 is also configured to receive a scattered signal (such as the RF signal transmitted from the RF transmitter) from a skin surface and to receive a scattered signal from deeper microvascular under skin in order to determine a bodily parameter, such as a heart rate signal.

Although FIGS. 2 and 3 illustrate examples of devices in a communication system, various changes may be made to FIGS. 2 and 3. For example, various components in FIGS. 2 and 3 could be combined, further subdivided, or omitted and additional components could be added according to particular needs. As a particular example, the processor 340 could be divided into multiple processors, such as one or more central processing units (CPUs) and one or more graphics processing units (GPUs). Also, while FIG. 3 illustrates the client device 300 configured as a wearable smartwatch, client devices could be configured to operate as other types of mobile or stationary devices. In addition, as with computing and communication networks, client devices and servers can come in a wide variety of configurations, and FIGS. 2 and 3 do not limit this disclosure to any particular client device or server.

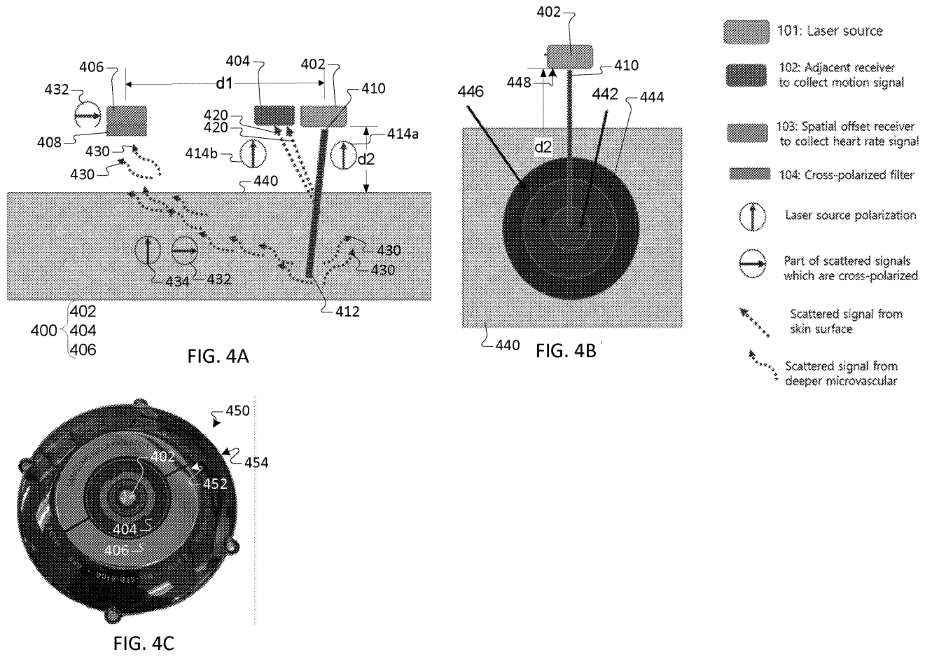

FIGS. 4A and 4B illustrate a side view and a top view of an example system diagram of a laser diffusion-based motion robust heart rate monitoring sensor 400 in accordance with an embodiment of this disclosure. FIG. 4C illustrates a bottom perspective view of a smartwatch 450 that includes hardware architecture of the laser diffusion-based motion robust heart rate monitoring sensor 400 in accordance with an embodiment of this disclosure. FIGS. 4A, 4B, and 4C are generally referred to as FIG. 4 in this disclosure. The embodiment of the sensor 400 shown in FIG. 4 is for illustration only, and other embodiments could be used without departing from the scope of this disclosure.

The sensor 400 includes specially-designed optical hardware that, when worn by a user, detects the human heart rate of the user. The heart rate can be detected by the sensor 400 while the human is exercising, for example, running or jumping. The sensor 400 can robustly detect the human heart rate of the user correctly without interference from a motion signal.

Referring to the side view of FIG. 4A, the sensor 400 includes a laser transmitter 402, a spatially adjacent laser receiver 404 that is located spatially adjacent relative to the laser transmitter 402, and a spatially offset laser receiver 406 that is located spatially offset relative to the laser transmitter 402. In this disclosure, the term “laser transmitter” is used interchangeably with the term “laser source.” The spatially adjacent receiver 404 is located next to the laser transmitter 402. The spatially offset laser receiver 406 is located away from the laser transmitter 402. The sensor 400 includes a cross-polarized filter 408 beneath the spatially offset receiver 406, and the cross-polarized filter 408 is used to filter out signals that share the same polarization as the laser transmitter 402.

The laser transmitter 402 emits a laser 410 that impinges skin of the user: a first portion of the laser 410 reflects off the surface of the skin thereby resulting in reflected signals 420 (referred to as return signals in this disclosure); a second portion of the laser 410 penetrates the skin to a penetration depth 412 that is at least as deep blood vessels deep under the skin, and a third portion of the laser 410 absorbs into the human body. The second portion of the laser 410, when incident upon the blood vessel, is likely to scatter with into different directions with various polarizations thereby resulting in scattered signals 430 (referred to as return signals in this disclosure).

The laser transmitter 402 transmits the laser 410 having a laser source polarization 414a. The skin surface 440 reflects a first portion of the laser 410 such that the spatially adjacent receiver 404 receives the reflections as reflected signals 420. The reflected signals 420 have the same laser source polarization 416 that the laser 410 has. The angles of reflection of reflected signals 420 relative to the skin surface 440 can be different from the angle of incidence of the laser 410 relative to the skin surface 440. That is, the spatially adjacent receiver 404 is not limited to detecting reflected signals 420 that travel in a direction antiparallel to the direction of the laser 410, as the location next to the laser transmitter 402 enables detection of from multiple angles of reflection within a range of angles.

When a human heart beats, the blood flow inside the blood vessel will vary which will result in a different absorptions of light signals when the light signals reach the blood vessel. Because the blood vessel is deep beneath the skin (for example, deep beneath the skin surface), a laser signal travels to the deeper layer under the skin is more likely to be able to reach the blood vessel. Due to the optical diffusion physics, a laser signal traveling deeper into the skin is more likely to scatter into different directions with various polarizations. At the penetration depth 412 of the laser 410, the laser 410 is incident upon one or more blood vessels such resulting in scattered signals 430. By scattering, the scattered signals 430 can have polarizations different from the laser source polarization 414a, such that part of the scattered signals 430 are cross-polarized 432 and another part of the scattered signals 430 are similarly polarized 434 like the laser source polarization 414a. For example, the cross-polarization 432 can be horizonal polarization, and vertical polarization can be associated with the laser source polarization 414a and the similarly polarized 434 part of the scattered signals.

As a reaction to the laser 410 impinging the blood vessel, the laser then carries (for example absorbs, or begins to embody) the heart rate information from the blood vessel into the scattered optical signal 430. In other words, the laser 410 impinging the blood vessel undergoes optical diffusion physics (such as scattering phenomenon) such that a collection of scatterers forms. The collection of scatterers can be represented as a total scattered wave, which is a vector sum of the scattered waves from each of the component scatterers. In this disclosure, the scatterers are referred to as blood flow signals, which are denoted as scattered signals 430.

These scattered signals 430 from deeper layer under the skin surface 440 carrying the heartbeat signal are more likely to reach the spatial offset receiver 406 located a distance d1 laterally farther away from the laser transmitter 402 than the spatially adjacent receiver 404. The scattered signals 430 received by the spatial offset receiver 406 are mostly carrying information of the heartbeat and heart rate. The scattered signals 430 are mainly modulated or changed by blood vessel movement, which is caused by the flow of blood inside the vessel according to the heartbeat. Naturally, the heartbeat and blood flow are closely (for example, causally) linked; and analogously, the heartbeat signal is closely linked to blood flow signals such that the blood flow signals include heart rate information.

During human body motion such as exercise, the relative distance d2 between laser transmitter 402 and the skin can vary, which changes the total optical path from the laser transmitter 402 to the skin and returning back from the skin to the spatially adjacent laser receiver 404. This change to the total optical path will result in a signal variation which carries the information of the human body motion. As laser signals (such as the laser 410) are shooting towards the skin surface 440 with a small incidence angle, the reflected signals 420 from the skin surface 440 are most likely to be received by the adjacent laser receiver 404 located laterally next to the laser transmitter 402 in close proximity. In the event that some of the reflected signals 420 (i.e., reflections) scatter at the skin surface 440, very few scattered reflections from skin surface 440 can travel far enough to reach the spatially offset laser receiver 406.

The laser light scattered from the skin surface 440 experiences less diffusion compared to the optical diffusion at the blood vessel at the penetration depth 412, and as a result, the polarization 414b of the reflected signal 420 is mostly the same as the laser source polarization 414a. In rare scenarios, such as when human body motion is large creating a very large incidence angle of the laser signal 410 at the skin surface 440, some of the reflected signal 420 from the skin surface reaches the spatial offset receiver 406, but the cross-polarization filter 408 filters out reflected signals 420. The cross-polarization filter 408 is cross-polarized 432, which means that the cross-polarization filter 408 has an orthogonal polarization direction to the polarization direction of the laser source polarization 414a.

Human body motion causes the smartwatch worn on the user's wrist to move relative to the user's wrist, especially during exercise. As a result, human body motion not only changes the incidence angle at which the laser 410 impinges the skin surface 440, but also, causes the distance d2 between a user's skin and the laser transmitter 402 to vary. The sensor 400 measures the human body motion by detecting one or more human motion signals using a spatially adjacent pair of laser transmitter and laser receiver (for example, laser transmitter 402 and the spatially adjacent laser receiver 404). In various embodiments, the one or more human motion signals can be the one or more reflected signals 420 received by the spatially adjacent laser receiver 404, can be features extracted from the one or more reflected signals 420, or can be features extracted from a set of motion data sensed by multiple motion sensors. The spatially adjacent pair of laser transmitter 402 and laser receiver 404 is one motion sensor among the multiple motion sensors. The reflected signals 420 are among the set of motion data.

The change of the total optical path between the laser transmitter 402 and a user's skin and the adjacent laser receiver 404 affects the return signals, which include the reflected signals 420 and the scattered signals 430, such that the return signals can actually change a lot. The reflected signals 420 are heavily impacted by the movement of the human body, but in comparison, the scattered signals 430 are mainly modulated or changed by blood vessel movement that is basically the heartbeat. The movement of the human body also impacts the return signals received by the spatially offset laser receiver 406, for example, in rare scenarios when some of the reflected signals 420 reach the spatial offset receiver 406 as a motion interference signal. The cross-polarized filter 408 rejects reflected signals 420 as motion interference signals.

Movement of the human body impacts the return signals received by the spatially offset laser receiver 406 in other ways. For example, human body movement interferes with the trajectory and/or speed of the scattered signals 430 from a blood vessel to the spatially offset laser receiver 406 and thereby introduces a motion interference signal to the blood flow signals. The motion interference signal introduced is a technical problem to which this disclosure provides a technical solution, which is to reduce (for example, cancel out) the motion interference signal within the blood flow signals by filtering the human motion signals (as detected by the spatially adjacent pair of laser transmitter 402 and laser receiver 404) out from the blood flow signals.

Referring to the top view of the skin surface 440 in FIG. 4B, the laser transmitter 402 emits a laser 410 in a first direction (for example, vertically downward) to a target such as a region of a human body. For ease of illustration and to avoid obfuscating the three concentric areas 442, 444, and 446, the laser transmitter 402 and emitted laser 410 are shown in a side view perspective while the skin surface 440 is shown in top view perspective. These three concentric areas 442-446 represent areas of the user's arm vertically underneath bottom surface 448 of the sensor 400, which is a distance d2 from the skin surface 440.

Surrounding (for example, concentrically about) the location where the laser 410 is incident upon the skin surface 440, there is a first area 442, a second area 444, and a third area 446. The third area 446 is where the spatially offset receiver 406 receives scattered signals 430 from deeper microvascular.

The first area 442 is where the reflected signals 420 originate at the skin surface 440. The first portion of the laser 410 that reflects off the first area 442 of the skin surface 440 divides into two sub-portions: a first sub-portion and a remainder sub-portion. The first sub-portion has reflection angles directed to the location of the spatially adjacent receiver 404, and the spatially adjacent receiver 404 receives this first sub-portion of the reflected signals 420 originating from the first area 442. The remaining sub-portion has reflection angles directed away from the location of the spatially adjacent receiver 404, for example, directed (radially or laterally) farther away than the outer perimeter of the spatially adjacent receiver 404.

A second area 444 extends radially from the outer perimeter of the first area 442 to an outer perimeter of the second area 444 that borders the inner perimeter of the third area 446. The second area 444 can represent a two-dimensional cross-section of a three-dimensional transition space that extends vertically from a bottom surface 428 of the sensor 400 to the penetration depth 412 beneath the skin surface 440. It is not impossible for some reflected signals 420 and some scattered signals 430 to propagate within the 3D transition space. However, the thickness of the second area 444 makes it unlikely that the adjacent receiver 404 receives a scattered signal 430, and reduces the probability of the offset laser receiver 406 receiving a reflected signal 420. The reflected signals 420 that successfully traverse across the thickness of the second area 444 (in the radial dimension) and reach the spatially offset receiver 406 will be filtered out (for example, rejected or discarded) by the cross-polarized filter 408.

Some embodiments include the physical 3D transition space such that the second area 444 extends radially from the outer perimeter of the spatially adjacent receiver 404 to the inner perimeter of the spatially offset laser receiver 406. That is, the spatially adjacent receiver 404 and the spatially offset laser receiver 406 are spatially offset from each other. The adjacent laser receiver 404 detects the human motion signal from the reflected signals 420 within the first area 442, and the spatially offset laser receiver 406 detects a heartbeat signal from the scattered signals 430 within the third area 446. The radius of the first area 442 can be designed to such that a majority of the reflected signals 420 impinge upon a plane of detection of return signals at radius less than the radius of the first area 442. The plane of detection of return signals can be coplanar with the bottom surface 448 of the sensor 400 that faces the skin surface 440. In some embodiments, the laser transmitter 402, spatial offset receiver 406, and spatially adjacent receiver 404 are coplanar at the bottom surface 448.

In other embodiments the second area 444 extends radially from the outer perimeter of the laser transmitter 402 to the outer perimeter of the spatially adjacent receiver 404. The radius of the first area 442 can be designed to such that a majority of the reflected signals 420 impinge upon a plane of detection of return signals (for example, coplanar with the bottom surface 448) at radius greater than the radius of the first area 442 and less than the radius of outer perimeter of the second area 444. For example, the offset distance d1 between the laser transmitter 402 and the offset receiver 406 does not require offset receiver 406 to be spatially offset from the spatially adjacent receiver 404. For example, the thickness of the second area 444 can be thinner than the thickness of the third area 446 in order to make it unlikely that the adjacent receiver 404 receives a scattered signal 430. For example, in order to reduce the probability of the offset laser receiver 406 receiving a reflected signal 420, the cross-polarized filter 408 can implement a functional transition space by filtering out the reflected signals that are closer to the laser transmitter 402 than the offset distance d1. The difference between these two embodiments is that the spatially adjacent pair laser transmitter 402 and laser receiver 404 (i.e., motion sensor) are both bounded within the first area 442 in the embodiment that includes the physical 3D transition space, but only the laser receiver 404 is bounded within the first area 442 in the embodiment that includes the functional transition space.

As shown in FIG. 4C, the bottom perspective view of the hardware architecture (including the spatial relationships and shapes) of the sensor 400 faces the skin surface 440. The laser transmitter 402 is positioned at the center of the smartwatch, and has a round shape similar to a coin. The adjacent laser receiver 404 has a ring shape that concentrically surrounds the laser transmitter 402. The spatially offset laser receiver 406 The laser transmitter 402 has a ring shape that concentrically surrounds the adjacent laser receiver 404.

The hardware architecture of the laser transmitter 402, spatially adjacent receiver 404, and spatial offset receiver 406 in FIG. 3C can be positioned at the first, second, and third areas 442, 444, and 446 of FIG. 4B, respectively. That is, laser transmitter 402 can have the round shape of the first area 442 or a different suitable shape. Instead of the first area 442 representing both the location of impingement of the laser 410 and the surrounding area through which the spatially adjacent receiver 404 receives reflected signals 420, the first area 442 can represent the laser transmitter 402 within a smartwatch.

The spatially adjacent receiver 404 can have the shape and location of the second area 444. The ring shape enables detection of reflected signals 420 in directions 360° around the laser transmitter 402. In some embodiments such as those including a functional transition space, the spatially adjacent receiver 404 occupies the entire radial range of the second area 444 such that its inner perimeter borders (for example, physically contacts or overlaps) the perimeter of the laser transmitter 402 and its outer perimeter borders the inner perimeter of the spatially offset laser receiver 406. In other embodiments such as those including a physical 3D transition space, the spatially adjacent receiver 404 occupies a partial of the radial range of the second area 444 by extending within a range of radiuses less than the inner radius of the second area 444.

Similarly, the spatially offset receiver 406 can have the shape and location of the third area 446. In some cases, the bottom surface 448 (FIG. 4B) of the sensor 400 can be the bottom surface of the smartwatch 450. Other vertical arrangements are possible, for example, the laser transmitter 402 can be recessed relative to one or both of the laser receivers 404 and 406. As another example, an outer perimeter 452 of the spatially offset laser receiver 406 at the bottom surface 448 (FIG. 4B) of the sensor400 can protrude closer to the skin surface 440 than and outer perimeter 454 of the smartwatch 450. The smartwatch 450 can include a beveled edge that extends between the outer perimeters 452 and 454 of the spatially offset laser receiver 406 and the smartwatch 450.

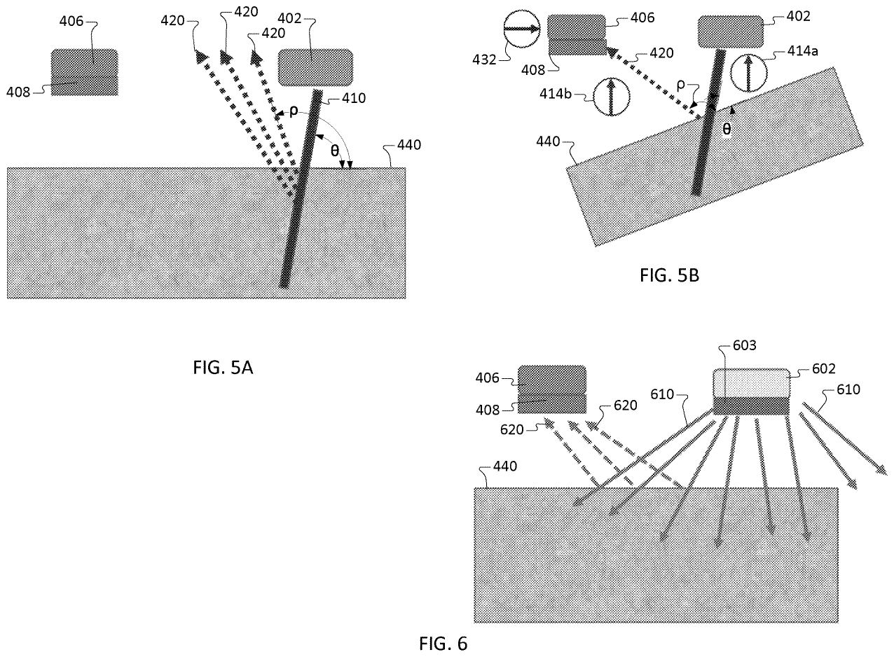

FIG. 5A illustrates an example usual scenario 500 of reflected signals 420 from the skin surface 440 exhibiting difficulty or inability to reach a spatially offset laser receiver 406 in accordance with an embodiment of this disclosure. FIG. 5B illustrates an example rare scenario 501 of reflected signals 420 from the skin surface 440 that reach a spatially offset laser receiver 406 in accordance with an embodiment of this disclosure. The embodiment of the reflected signals 420 shown in FIGS. 5A and 5B are for illustration only, and other embodiments could be used without departing from the scope of this disclosure. As a similarity among FIGS. 5A and 5B, the reflected signal 420 from skin surface 440 has a polarization 414b that is the same as the laser source polarization 414a associated with the laser 410.

A comparison of FIGS. 5A and 5B demonstrates that the incidence angle θ of the laser 410 relative to the skin surface 440 controls whether the reflected signal 420 is among the return signals that the cross-polarized filter 408 on the spatially offset laser receiver 406 receives as input. The reflected signals 420 are mostly likely to reflect at reflection angles ρ directed to an area immediately surrounding the laser transmitter 402 when the incidence angle θ of the laser 410 is a small incidence angle, which is when an angular difference between the incidence angle θ and a 90° angle of an axis orthogonal to the skin surface 440 is small, as shown the usual scenario 500 of FIG. 5A. The area immediately surrounding the laser transmitter 402 is the location of the spatially adjacent receiver 404 (hidden for ease of illustration).

The cross-polarized filter 408 does not receive any reflected signals 420 that have small reflection angles ρ as shown in the usual scenario 500 of FIG. 5A. However, the cross-polarized filter 408 receives the reflected signal 420 that has a very large reflection angle ρ as shown in the rare scenario 501 of FIG. 5B. In the rare scenario 501 of FIG. 5B, the reflected signal 420 will be filtered out by the cross-polarized filter 408 on the spatially offset laser receiver 406.

FIG. 6 illustrates a non-laser diffusion-based motion robust heart rate monitoring sensor 600 that includes a light emitting diode (LED) light source 602. The LED light source 602 performs inferiorly compared to the laser transmitter 402 of FIG. 4. The laser transmitter 402 of FIG. 4 is a unidirectional narrow beam, naturally polarized, and penetrates deeper into skin than LED light due to a focused beam (for example, having same frequency, power).

In comparison, the LED light source 602 is not naturally polarized. A light source polarization filter 603 is combined with or attached to the LED light source 602 to generate a light polarization source, which can have a polarization similar to the laser source polarization 414a of FIG. 4. A higher polarized source signal can lead to a better signal to noise ratio (SNR).

The LED signal 610 is omnidirectional, compared to the laser 410 that is unidirectional. That is, the LED signal 610 reflects at the skin surface, thereby forming LED reflections 620 that have a high chance of reaching the spatial offset receiver 606. The angles of reflection of the LED reflections 620 can be large and directed farther away than a spatially adjacent receiver 404, even when a 90° angle of an axis orthogonal to the LED light source 602 is also orthogonal to the skin surface 440.

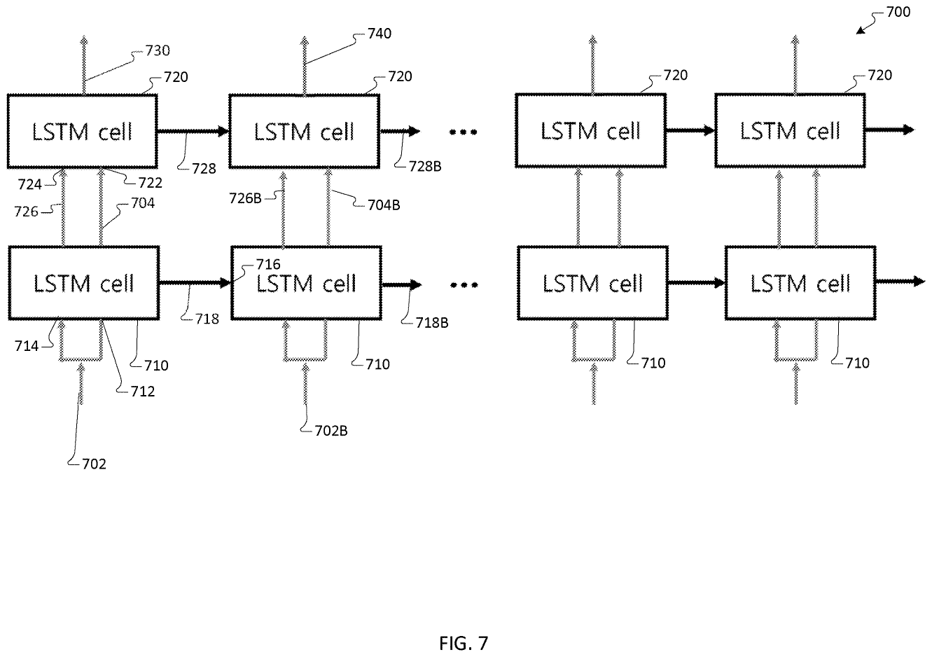

FIG. 7 illustrates an example sequential signal processing (SSP) model 700 in accordance with an embodiment of this disclosure. The embodiment of the SSP model 700 shown in FIG. 7 is for illustration only, and other embodiments could be used without departing from the scope of this disclosure. The SSP model 700 can be stored in a memory and executed by a processor of a wearable device, such as the memory 360 and processor 340 of FIG. 3. The SSP model 700 is a sequential artificial intelligence (AI) model for robust heart rate monitoring.

The SSP model 700 periodically receives a motion filtered heartbeat signal 702 output by the offset laser receiver 406, such as at time t1, t2, t3, and t4. The motion filtered heartbeat signal 702 can be the same as or similar to the scattered signals 430 after passed through the cross-polarized filter 408.

The SSP model 700 periodically receives an estimated motion signal 704 output by the adjacent laser receiver 404, such as at time t0, t1, t2, and t3. The estimated motion signal 704 can be the same as or similar to the reflected signals 420.

After acquiring the motion filtered heartbeat signal 702 and the estimated motion signal 704 from the offset and adjacent receivers 406 and 404, respectively, the SSP model 700 is applied for robust heart rate monitoring. The architecture of the SSP model 700 includes a double stacked long short term memory (LSTM) model. The double stacked LSTM model includes a first LSTM cell 710 that receives the motion filtered heartbeat signal 702 for a current time, such as timestamp t0. The first LSTM cell 710 can include a first input terminal 712 associated with receiving an input measurement from the spatially offset laser receiver 406, and a second input terminal 714 that receives the same input measurement that the first input terminal receives. The first LSTM cell 710 extracts and outputs a first set of machine-learning (ML) features 726 configured for input into the second LSTM cell 720, based on the motion filtered heartbeat signal 702.

The first LSTM cell 720 also extracts and outputs a second set of ML features 718 configured for input into a feedback channel 716 of the first LSTM cell 710. The second set of ML extracted features 718 are extracted based on the motion filtered heartbeat signal 702. The second set of ML extracted features 718 are configured to improve a subsequent heart rate estimation 740 for a next time, such as timestamp t1.

The double stacked LSTM model includes a second LSTM cell 720 that receives the motion signal 704 for a current time, such as timestamp t0. The second LSTM cell 720 includes a third input terminal 722 associated with receiving an input measurement from the spatially adjacent laser receiver 404, and a fourth input terminal 724 that receives the first set of ML extracted features 726 that are configured to improve a current heart rate estimation 730 when processed through the second LSTM cell 720. The second LSTM cell 720 generates current heart rate estimation 730 associated with timestamp to based on both the motion signal 704 and the first set of ML extracted features 726.

The second LSTM cell 720 also extracts and outputs a third set of ML features 728 configured for input into a feedback channel 716 of the second LSTM cell 710. The third set of ML extracted features 728 are extracted based on both the motion signal 704 and the first set of ML extracted features 726. The third set of ML extracted features 728 are configured to improve a subsequent heart rate estimation 740 for a next time, such as timestamp t1.

To train the AI-based SSP model 700, the motion filtered heartbeat signals 702 and motion signal 704 from the sensor 400 are collected along with a ground truth heart rate, which can be measured in beats per minute (BPM). Then the AI-based SSP model 700 is trained with backpropagation. After training, the trained AI-based SSP model 700 estimates a heart rate periodically, such that a sequence of heart rate values can be estimated from the measured motion signal 704 and motion filtered heartbeat signal 702.

Sequentially, when timestamp t1 becomes the current time, the double stacked LSTM model is reapplied. The first LSTM cell 710 receives a motion signal 702b for the current time corresponding to timestamp t1. Based on the second set of ML extracted features 718 received as feedback, the first LSTM cell 710 processes the motion signal 702b to extract and output a fourth set of ML features 726b and a fifth set of ML features 718b. The fourth set ML extracted features 726b are configured for input into the second input terminal of the second LSTM cell 720.

The second LSTM cell 720 generates current heart rate estimation 730b associated with timestamp t1 based on: the motion signal 704b, the fourth set of ML extracted features 726, and the third set of ML extracted features 728 received as feedback. The second LSTM cell 720 also generates a sixth set of ML extracted features 728b.

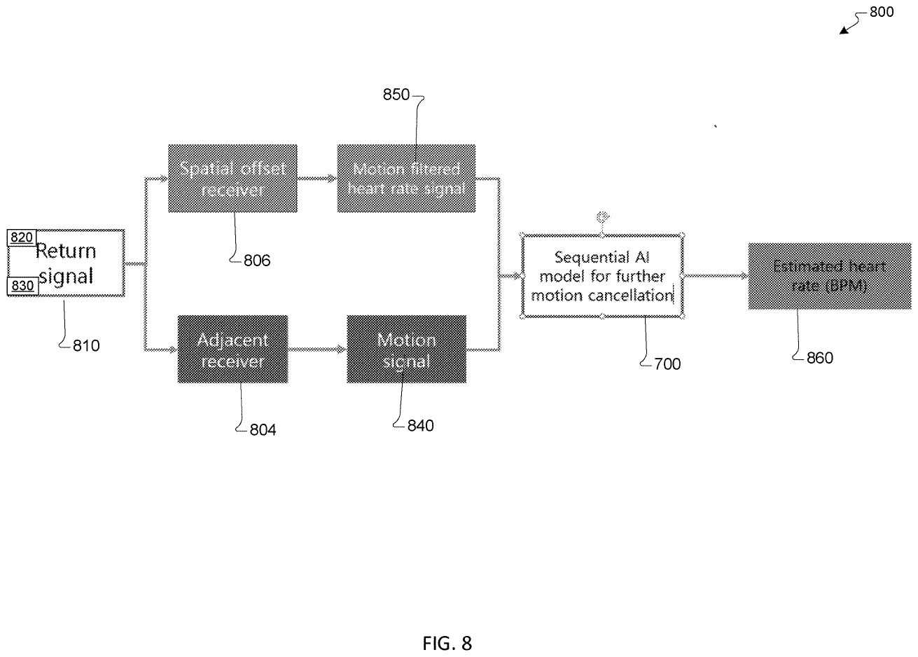

FIG. 8 illustrates an example signal processing operation 800 of the SSP model 700 of FIG. 7 for robust heart rate estimation in accordance with an embodiment of this disclosure. The embodiment of the operation 800 shown in FIG. 8 is for illustration only, and other embodiments could be used without departing from the scope of this disclosure. The operation 800 can be executed by a processor of a wearable device of FIG. 3.

The adjacent receiver 804 and spatial offset receiver 806 of FIG. 8 can be the same as or similar to the corresponding receivers 404 and 406 of FIG. 4. The return signals 810 can be a mixture of reflected signals 820 and scattered signals 830, which can be the same as or similar to: a mixture of the reflected signals 420 and the scattered signals 430 of FIG. 4, or a mixture of the motion filtered heartbeat signal 702 and estimated motion signal 704 of FIG. 7.

As one processing channel, the adjacent receiver 804 generates and outputs a motion signal 840 based on return signals 810 that the adjacent receiver 804 received. The return signals 810 that the adjacent receiver 804 received are predominantly the reflected signals 820 based on the hardware architecture of the sensor, and as a result, the motion signal 840 is unlikely to contain interference from blood flow signals. The motion signal 840 estimates or measures human body motion, and can be the same as or similar to the estimated motion signal 704 of FIG. 7.

As another processing channel, the offset receiver 806 generates and outputs a motion filtered heartbeat signal 850 that the offset receiver 806 received. The return signals 810 that the offset receiver 806 received are a mixture of predominantly a heartbeat signal (i.e., blood flow signals) 830 and a little of the reflected signals 820. As a result of the composition of the return signals 810 that the offset receiver 806 received, a motion interference signal is embedded with the motion filtered heartbeat signal 850.

The sequential AI model for further motion cancellation shown in FIG. 8 can be the SSP model 700 of FIG. 7. The SSP model 700 receives the motion-filtered heartbeat signal 850 into the first LSTM cell 710, receives the motion signal 840 into the second LSTM cell 720, and generates an estimated heart rate 860 corresponding to a current time. The estimated heart rate 860 output from the SSP model 700 in FIG. 8 can be the same as or similar to the estimated heart rate 730 or 740 of FIG. 7. The estimated heart rate 860 output from the SSP model 700 can be iteratively estimated to generate a sequence of heart rate estimates for timestamps t0-t3 and farther into the future.

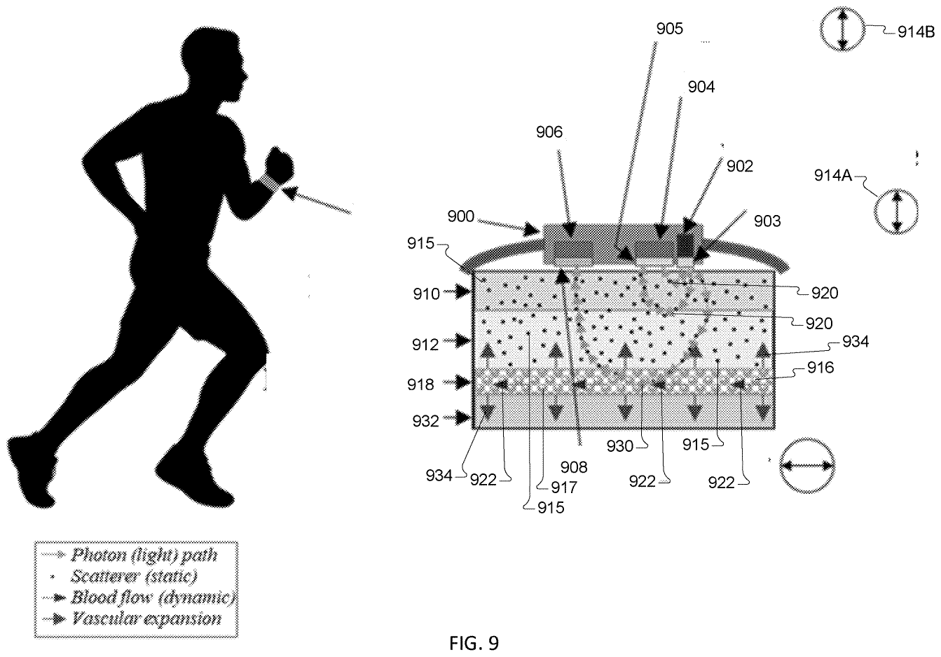

FIG. 9 illustrates an example a wearable device 950 including an integrated optical sensor (IOS) 900 for measuring human vital signs in accordance with an embodiment of this disclosure. The embodiment of the wearable device 950 and the IOS 900 shown in FIG. 9 are for illustration only, and other embodiments could be used without departing from the scope of this disclosure. A first hardware architecture of the IOS 900 is configured for generating motion artifact-free speckle plethysmographic (SPG) waveforms.

The IOS 900 operates primarily based on laser speckle imaging (LSI). The wearable device, such as a wristwatch, a ring, or an ear-pod, contains an IOS 900 for measuring human parameters including cardiovascular and respiratory signals such as heart rate, blood pressure, blood flow, respiratory rate, etc. during vigorous activities. The IOS 900 includes a laser diode 902 (e.g. a VCSEL laser) of a suitable wavelength, such as 785 nm, that propagates at least up to the blood vessels layer under the skin. A polarizer 903 may be affixed to the window (or opening or entrance) of the laser diode 902 to ensure that the light emerging from the laser is of a specific polarization, such as vertical polarization 914a. Alternatively, the intrinsic (or natural) polarization of the laser may be utilized. As the laser light propagates through the different layers of the skin and tissues, it gets randomly scattered multiple times by a combination of static and dynamic scatters.

The static scatterers 915 include the extracellular matrix or extracellular macromolecules and minerals that provide structural and biochemical support to surrounding cells like collagen, etc., and stationary cells. The membranes belonging to the moving red blood cells constitute the dynamic scatters 916. In addition to random scattering, the light also undergoes attenuation as it passes through the tissues due to absorption by hemoglobin in the red blood cells (and other absorbers in the tissues). While the dynamic scattering phenomenon can be used to infer volumetric blood flow, the variation in the light absorption caused the variation in the concentration of absorbers mirrors the vascular expansion during cardiac cycle. These two pulsatile signals, originating from distinct physiological processes, can provide useful information about heart rate, blood pressure, etc. Some of the scattered light propagates backwards to IOS 900. The vascular expansion direction 934 occurs in a radially outward direction relative to the blood vessels 918. The vascular expansion direction 934 can be orthogonal to the direction of blood flow 922.

The IOS 900 includes at least two polarization-selective detectors 904 and 906 that operate at a high frame rate, such as greater than 200 frames per second. The first detector 904 is placed adjacent to the laser diode 902, such as within approximately a couple of millimeters. A co-polarizer 905 is placed at the entrance of the first detector 904. The co-polarizer 905 is a polarization filter having the same polarization 914b as the polarizer 903 of the laser diode 902, for example, vertical polarization.

The second detector 906 is placed farther away from the laser diode 902, for example, approximately five millimeters to within a few centimeters, which can be the same as or similar to the separation distance d1 of FIG. 4. A cross-polarizer 908, whose orientation is orthogonal to that of the laser, is placed at the entrance of the second detector 906. Most of the backscattered light that reaches the first detector 904 are from the superficial layers 910 of the skin and retain the same polarization state due to fewer scattering. The superficial layers 910 of the skin include the epidermis, which reflects or scatters light at a shallow depth.

The co-polarizer 905 ensures that light (such as a light signal emitted from the laser diode 902) that has undergone only few scattering events from shallow depths, such as one or more shallow photon paths 920, reaches the first detector 904. This light signal of the shallow photon paths 920 never interacts with the deeper layers 912 of skin (for example, dermis layer) and blood vessels 918, and as a result, a shallow photon path 920 does not carry any blood flow related physiological information. However, in the presence of relative-motion between the IOS 900 and the skin (or human body), this relative-motion information will be embedded in this light signal of the shallow photon paths 920. The co-polarizer 905 ensures that only slightly scattered light, from the shallow layers 910 can reach this first detector 904.

The second detector 906 is farther away from the laser diode 902 light source, and as a result, the second detector 906 receives the light of the deep photon paths 930 that has travelled through the deeper layers 912 of the dermis and blood vessels 918 after interacting with dynamic scatters 916 such as the membranes belonging to moving red blood cells. The direction of blood flow 922 is shown within the layer of blood vessels 918. The several scattering events along the deep photon path 930 depolarizes this light. A fat layer 932 can be a deeper layer than the layer of blood vessels 918.

The cross-polarizer 908 at the entrance of the second detector 906 filters out most of the light backscattered from the superficial layers 910. For example, the cross-polarizer 908 can filter out light that has the same polarization as the polarizer 903, such as vertical polarization 914a. The output of the cross-polarizer 908 is the signal that the second detector 906 receives as input, which contains the physiological signal of interest along with motion information (such as motion interference signal).

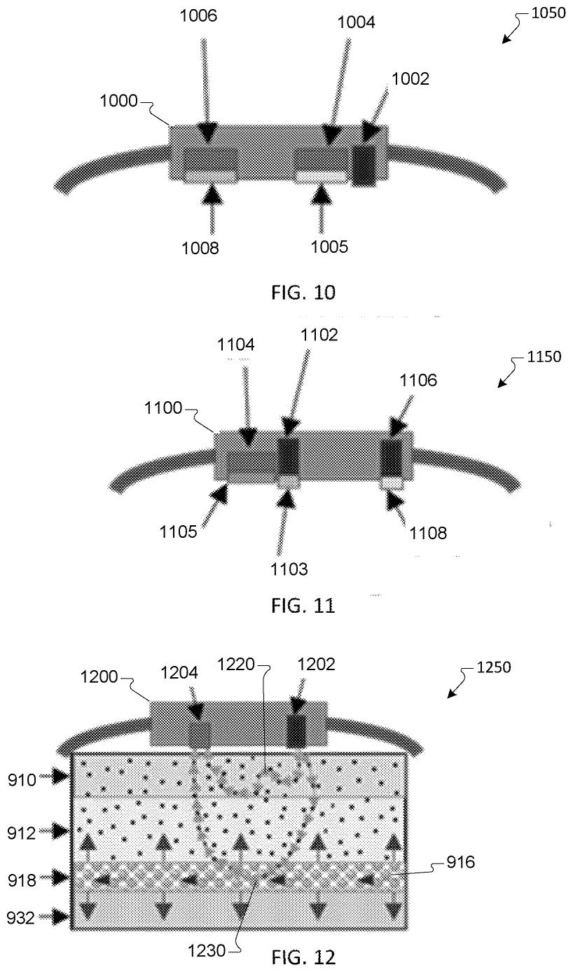

FIG. 10 illustrates an example a wearable device 1050 including a second hardware architecture of an integrated optical sensor (IOS) 1000 for measuring human vital signs in accordance with an embodiment of this disclosure. The embodiment of the second hardware architecture of the IOS 1000 shown in FIG. 10 is for illustration only, and other embodiments could be used without departing from the scope of this disclosure. The second hardware architecture of the IOS 1000 is configured for generating motion artifact-free speckle plethysmographic (SPG) waveforms.

The IOS 1000 includes a vertical-cavity surface-emitting laser (VCSEL) laser transmitter 1002, a first detector 1004 positioned spatially adjacent to the laser transmitter 1002, and a second detector 1006 positioned spatially offset from the laser transmitter 1002. The VCSEL laser transmitter 1002 emits polarized light of a specific polarization (such as vertical polarization). A polarizer is not affixed to the window (or opening) of the VCSEL laser transmitter 1002, which is a difference compared to the first hardware architecture of FIG. 9.

The first and second detectors 1004 and 1006 of FIG. 10 can be the same as or similar to the first and second detectors 904 and 906 of FIG. 9. Both detectors 1004 and 1006 have spatial light modulators (SLM) at their entrances, including a first SLM 1005 and a second SLM 1008, respectively. The SLMs can be used to control the polarization of light that is sensed at each detector.

FIG. 11 illustrates an example a wearable device 1150 including a third hardware architecture of an integrated optical sensor (IOS) 1100 for measuring human vital signs in accordance with an embodiment of this disclosure. The embodiment of the third hardware architecture of the IOS 1100 shown in FIG. 11 is for illustration only, and other embodiments could be used without departing from the scope of this disclosure.

The third hardware architecture of the IOS 1100 is configured for generating motion artifact-free SPG waveforms by employing two laser diodes 1102 and 1106 with orthogonal polarizations and a single detector 1104, instead of using two detectors in comparison to the hardware architectures of the sensors 400, 900, and 1000 of FIGS. 4, 9, and 10. A first laser diode 1102 is placed closer to the detector 1104 and the second laser diode 1106 placed spatially farther (for example, in the order of centimeters) from the detector 1004. As such, the first laser diode 1102 and the single detector 1104 form an adjacent offset pair of laser transmitter and laser receiver, and operate as a motion sensor that generates a motion signal. The second laser diode 1106 and the single detector 1104 form a spatially offset pair of laser transmitter and laser receiver, and operate to generate a blood flow signal.

In this hardware configuration, an SLM 1105 is placed at the entrance of the single detector 1104. The polarization state of the SLM 1105 changes at the frame rate of the detector such that the detector 1104 records the interference speckle patterns from the first laser diode 1102 and second laser diode in alternate frames. In addition to the practical advantage of the reduction of cost by using a single detector 1104 instead of multiple detectors, an additional benefit of the third hardware architecture shown in FIG. 11 is that the power of the two laser diodes 1102 and 1106 can be individually modulated to match the average intensities of the speckle patterns from the two.

A polarizer is placed at the window of one laser diode from among the first and second laser diodes 1102 and 1106, and a cross-polarizer is placed at the window of the other laser diode from among the two laser diodes 1102 and 1106. In this example, the first laser diode 1102 and its polarizer 1103 form a first VCSEL laser transmitter, and the second laser diode 1106 and its cross-polarizer (such as SLM 1105) form a second VCSEL laser transmitter. The polarization state of the SLM 1105 corresponds to the polarization (such as vertical polarization) of the first VCSEL laser transmitter in one frame capture, and next the polarization state of the SLM 1105 switches to correspond to the orthogonal polarization (such as horizontal polarization) of the second VCSEL laser transmitter in the next frame capture. A processor, such as the processor 340 of FIG. 3, can control the switching of the polarization state of the SLM 1105.

FIG. 12 illustrates an example a wearable device 1250 including a fourth hardware architecture of a time-domain diffusion correlation spectroscopy (TD-DCS) based robust heart rate monitoring sensor 1200 in accordance with an embodiment of this disclosure. The embodiment of the third hardware architecture of the IOS 1100 shown in FIG. 11 is for illustration only, and other embodiments could be used without departing from the scope of this disclosure.

The legend shown in FIG. 9 applies as the legend of FIG. 12. The bodily tissue layers 910, 912, 918, and 932, the static scatterers 915, and dynamic scatterers 917, and directions of vascular expansion included in the cross-section of bodily tissue of FIG. 9 are also shown in FIG. 12.

The fourth hardware architecture of the sensor 1200 is configured for motion artifact-free blood flow measurement using TD-DCS. This TD-DCS is a variation of diffusion correlation spectroscopy (DCS) that offers improved depth sensitivity and the ability to separate signals from different tissue layers. The two main hardware components for TD-DCS-based blood flow measurement are a laser transmitter 1202 that is a pulsed VCSEL laser source 1202 and a single detector 1204 that is a single-photon avalanche diode (SPAD) which detects individual photons arriving at the detector 1204. The laser transmitter 1202 can operate in a similar manner as the laser transmitter 402 of FIG. 4. More particularly, laser transmitter 1202 emits pulsed light.

The TD-DCS technology utilizes pulsed light and measures the time-of-flight (TOF) of photons as the photons propagate through the bodily tissue. Photons scattered from superficial layers, such as the epidermis, take shorter, more direct paths to the detector and thus have shorter times of flight (TOFs). Conversely, photons that penetrate deeper, like those reaching the lower dermis and blood vessels experience more scattering, travel longer distances, and consequently exhibit longer TOFs. Time-gating, which involves defining specific time windows known as “gates,” is used to select the photons arriving within a specific time window (for example, a 0-100 picosecond gate for superficial layers). Photons arriving within that specific time window are analyzed. In some embodiments, photons arriving outside that specific time window are discarded or not processed further. Thus, time-gating enables depth selectivity control in TD-DCS. The early-arriving photons are more representative of the superficial tissue layers. Similarly, photons arriving in later time windows are considered late-arriving photons, and contain information from deeper tissue regions.

When a photon arrives at a SPAD detector, the arrived photon triggers a rapid avalanche of electrons, thereby creating a pulse of electrical current. In addition, SPAD detectors have high temporal resolutions enabling them to detect and time the arrival of photons with picosecond accuracy, enabling precise TOF measurements.

In order for the computer processor to separate and separately analyze the early-arriving photons and late-arriving photons, TD-DCS is used to calculate intensity autocorrelation functions for photons falling within each specified time gate. The autocorrelation function provides information about the dynamics of the scatterers 916 (red blood cells) within the tissue, quantified as a blood flow index (BFI). By separately calculating the intensity autocorrelation functions for each of the time gates, TD-DCS effectively isolates the signals from the superficial and deep layers from each other. In other words, the early-arriving photons reflect or scatter within the superficial layers 910 along the shallow photon paths 1220 are isolated from and analyzed separately from the late-arriving photons reflect or scatter within the deeper layers 912 and blood vessels 918 along the deeper photon paths 1230.

While the signal from shallow photon paths 1220 of the superficial layer 910 mainly contains human body movement information, the signal from deeper photon paths 1230 of the deeper layers contain blood flow information along with motion (if any motion interference is present). Analogous to the PPG and SPG, the BFI signal is a time-varying waveform representing changes in blood flow within the bodily tissue being measured. The BFI signal exhibits pulsatile oscillations and these pulsations are often synchronized with a heartbeat and represent the rhythmic changes in blood flow caused by the cardiac cycle.

Once the BFI signals (which is possibly affected by movement artifacts) and the movement signal has been obtained from the two bodily tissue layers 910 and 918, the motion in the BFI signal is suppressed using an appropriate motion suppression technique discussed previously. For example, the SSP model 700 of FIGS. 7-8 can be applied return signals including: the early-arriving photons along the shallow photon paths 1220 that can be input to the first LSTM cell 710; and the later-arriving photons along the deeper photon paths 1230 that can be input to the second LSTM cell 720.

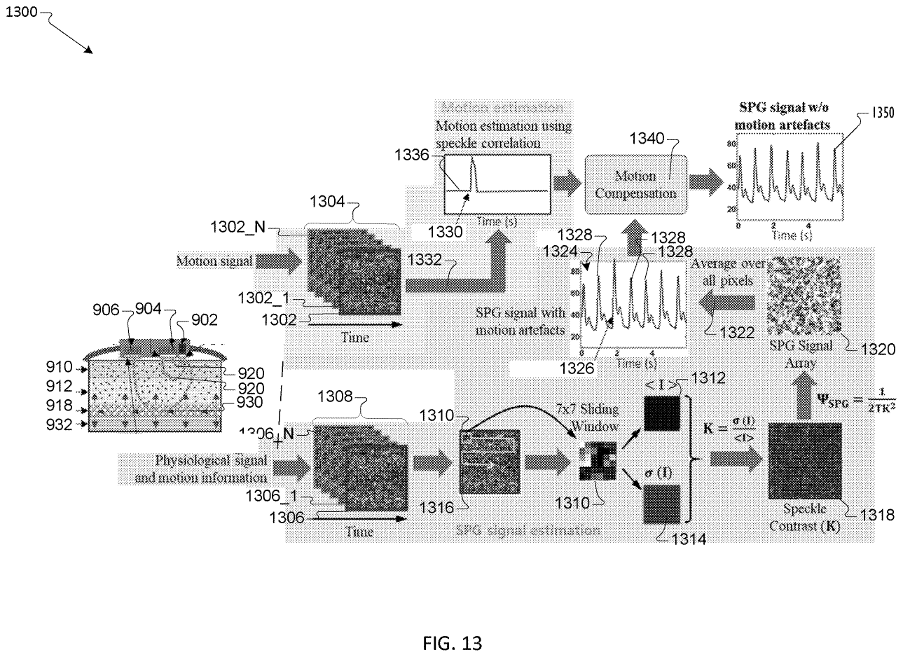

FIG. 13 illustrates an example process 1300 for generating a motion artefact-free SPG signal from two two-dimensional detectors in accordance with an embodiment of this disclosure. The embodiment of the process 1300 shown in FIG. 13 is for illustration only, and other embodiments could be used without departing from the scope of this disclosure.

The legend shown in FIG. 9 applies as the legend of FIG. 13. The bodily tissue layers 910, 912, 918, and 932, the static scatterers 915, and dynamic scatterers 917, and directions of vascular expansion included in the cross-section of bodily tissue of FIG. 9 are also shown in FIG. 13. For ease of explanation, the process 130 of FIG. 13 will be described as processing signals output by the sensor 900 within the wearable device 950 of FIG. 9, however, it is understood that process 1300 can be applied to process signals output by the sensor 1000 of FIG. 10 or by the IOS 1100 of FIG. 11.

SPG measures blood flow dynamics by analyzing the changes in the random speckle pattern generated from light dynamically scattered by moving dynamic scatters 916, such as blood cells, within bodily tissue. As a technical advantage, SPG provides higher signal-to-noise ratio compared to PPG and is less susceptible to variations in ambient light and skin melanin content.

The process 1300 operates based on an assumption that within the sensor 900, each of the two detectors 904 and 906 is a two-dimensional sensor, such as CMOS or CCD image sensor sensitive to the wavelength of the laser source of the laser transmitter 902. The back-scattered coherent light generates speckle interference patterns on the two detectors 904 and 906.

The first detector 904 generates a speckle 1302 based on the interference of light scattered mostly from the static scatters 915 in the superficial layers 910 of skin and tissue. The first detector 904 can capture a speckle periodically, for example, a time series 1304 of two-dimensional captures by the first detector 904 can be a set of correlated speckles from the epidermis. The time series 1304 includes the current speckle 1302 captured at a current time to, and previously captured speckles 1302_1 through 1302_N. In the absence of any human body motion, the speckle pattern remains relatively fixed and stable over time.

On the other hand, in the event of any relative motion between the IOS 900 and body (skin and tissue) or group motion of the static scatters 915 due to tissue stretching, the speckle pattern captured by the first detector 904 will shift spatially reflecting the underlying motion due to the memory effect of speckle patterns. A high frame rate detector enables the process 1300 to detect the shifting spatially due to memory effect of speckle patterns. If a high frame rate detector is not used, instead of shifting spatially due to memory effect, the speckle pattern will exhibit the boiling effect. By correlating the speckle patterns frame-by-frame, the motion, if any can be accurately estimated.

The second detector 906 generates a speckle 1306 corresponding to a current time t0. A time series 1308 of two-dimensional captures by the second detector 906 can be a set of non-correlated speckles from the dermis deeper layer 912 and blood vessels 918. In the absence of any physical motion, the speckle 1306 generated at the second detector 906 fluctuates mainly (for example, only) in response to the relative blood flow 922. The relative blood flow 922 is the pulsatile movement of the red blood cells and the pulsatile vascular expansion. In the absence of any physical motion, the fluctuations in the speckle pattern and the overall blurring of the individual speckles (among the current speckle 1306 and previous speckles 1306_1 through 1306_N) encode the pulsatile blood flow corresponding to the cardiac cycle. As a result, the relative blood flow can be measured from the dynamic speckle contrast. The heart rate (i.e., heart pulse rate) and other physiological indicators or vital signs can then be derived from the blood flow.

The process 1300 includes a procedure for deriving the SPG from the de-correlated and pulsatile speckle pattern among the time series 1308. To obtain the SPG signal, the process begins by shining a coherent light source (such as laser diode 902) on a part of the body—for example, on the wrist if the IOS 900 is part of a smartwatch. The backscattered light from the deeper layers is captured by the second detector 906 that records the dynamic speckle interference pattern as a series 1308 of frames.

For each captured frame, a sliding window filter 1310 (for example of size 7×7 pixels) is applied to determine the average intensity (<I>) 1312 and the standard deviation (σ(I)) 1314 within that sliding window filter 1310. Although the window-based averaging reduces the spatial resolution of the speckle image, it is a technically advantageous step to preserve the assumption of first order speckle statistics. The sliding window slides in a specified path 1316, such as a start point at the upper-left pixel of the current speckle 1306 frame, sliding horizontally rightward, sliding vertically down, sliding vertically down, and then repeating this cycle.

A speckle contrast matrix (K) array 1318 is calculated as the ratio of the standard deviation σ(I) array to the average intensity <I>, array. That is,

$$K = \frac{σ ( I )}{〈 I 〉} .$$

The SPG signal array (Ψ_{SPG}) 1320 is then obtained as

$${Ψ}_{SPG} = \frac{1}{2 {TK}^{2}} ,$$

where T is the integration time of the second detector 906.

Finally, the process 1300 includes applying an averaging function 1322 over all pixels of the SPG signal array (Ψ_{SPG}) 1320. That is, the SPG signal array 1320 is averaged to produce a single SPG signal value for that specific time point. By repeating this procedure for deriving the SPG from the de-correlated and pulsatile speckle pattern among the time series 1308 for every captured frame, a time-varying SPG waveform 1324 is constructed. This SPG waveform 1324 (including motion artifacts) reveals the pulsatile blood flow characteristics within the tissue.

The photoplethysmography (PPG) signal can also be obtained from the same raw data (such as the time series 1308) by measuring the average intensity variations resulting from pulsatile absorption of light. In the presence of physical motion of the IOS 900 relative to the human body or due to the group motion of the static scatters 915 as a result of tissue stretching during vigorous motion of the user's body, the generated SPG waveform 1324 (and PPG signal) will also contain the motion information along with the physiological relative blood flow information. Such undesirable and spurious artifacts 1326 due to motion typically results in inaccurate measurement of vital signs (e.g., heart pulse rate, respiratory rate, blood pressure, etc.). The spurious motion artifact 1326 can be an anomalously greater peak relative to recent peaks 1328 that are not affected by the increased magnitude of a motion interference signal.

In an embodiment of this process 1300, the motion artifacts 1326 can be greatly suppressed using the motion suppression techniques described in this disclosure. More specifically, the process 1300 includes a motion estimation procedure in which an extraneous motion 1330 (such as the same motion that caused the artifact 1326) is characterized by applying the speckle correlation function 1332. That is, during extraneous motion 1330, the speckle pattern observed at the first detector 904, shifts spatially over time in response to the underlying motion, which generates the motion estimation signal 1336 as one-dimensional data relative to time. The motion estimation procedure includes applying the image/speckle correlation function 1332 over frames among the time series 1304 the direction and magnitude of motion (provided the assumption of the memory effect of speckle patterns is not violated by ensuring a high frame rate).

Once the motion estimation signal 1336 (for example, a motion signal envelop is estimated), a motion suppression procedure 1340 is applied. There can be several techniques of suppressing the motion artifact in the SPG (and PPG) signal. A simple technique is to subtract or isolate the motion signal envelop (with proper normalization and biasing to zero or one to represent the motionless state) from the estimated SPG (and PPG) waveform 1324. The normalization factor may be derived based on a one-time factory calibration. Alternatively, another technique is a simple adaptive algorithm or AI-based motion compensation algorithm as described in other embodiments of this disclosure, such as applying the SSP model 700 of FIG. 7 to remove the effect of motion in the SPG (and PPG) signal.

The output from the motion suppression procedure 1340 is a heartbeat signal 1350, which can be post-processed to determine the heart rate of the use. In the process 1300, the heartbeat signal 1350 is the SPG signal without motion artifacts.

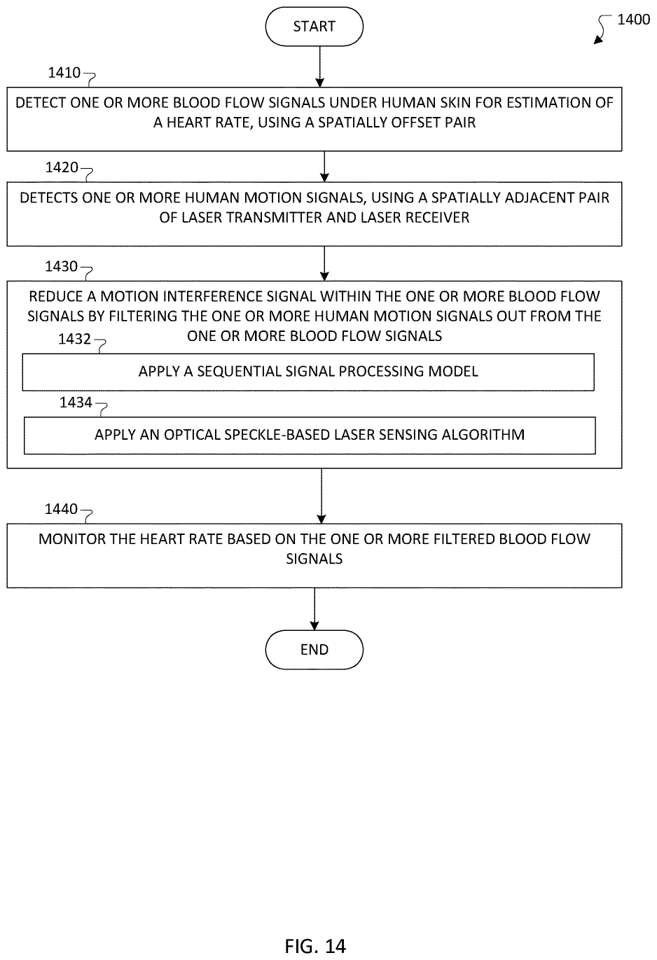

FIG. 14 illustrates a method 1400 for laser sensor system for direct heart rate signal measurement without interference from human body motion in accordance with an embodiment of this disclosure. The embodiment of the method 1400 shown in FIG. 14 is for illustration only, and other embodiments could be used without departing from the scope of this disclosure. The method 1400 is implemented by an electronic device, such as the electronic device 300 of FIG. 3. More particularly, the method 1400 could be performed by a processor 340 of the electronic device 300 executing the application 362. In some embodiments, within the spatially adjacent pair laser transmitter and laser receiver, the laser receiver includes a polarized filter such as polarized filter 905 of the adjacent detector 904 in FIG. 9.

In block 1410, the processor 340 detects one or more blood flow signals under human skin for estimation of a heart rate, using a spatially offset pair of laser transmitter and laser receiver. In some embodiments, within the spatially offset pair of laser transmitter and laser receiver, the laser receiver includes a cross-polarized filter, such as the two SLMs 1103 and 1105 that function as the polarizer and cross-polarizer that are cross-polarized relative to each other and one or which is located at the window (openings) of the laser transmitter 1106 spatially offset from the single detector 1104 as shown in FIG. 11. In some embodiments, as shown in FIG. 11, the spatially offset pair of laser transmitter and laser receiver includes a first laser transmitter 1106 that includes a polarizer 1108 and a first laser receiver (such as single detector 1104); and the spatially adjacent pair laser transmitter and laser receiver includes a second laser transmitter 1102 that includes a cross-polarizer 1103 and the first laser receiver (such as single detector 1104).

In block 1420, the processor 340 detects one or more human motion signals, using a spatially adjacent pair of laser transmitter and laser receiver. For example, the one or more human motion signals can be the reflected signals 420 that the spatially adjacent receiver 404 receives in FIG. 4. As another example, the one or more human motion signals can be light signal of the shallow photon paths 920 that the first detector 904 receives in FIG. 9, or that the first detector 1004 receives in FIG. 10. As another example, the one or more human motion signals can be the early-arriving photons along the shallow photon paths 1220 in FIG. 12.

In block 1430, the processor 340 reduces a motion interference signal within the one or more blood flow signals by filtering the one or more human motion signals out from the one or more blood flow signals. In other words, the processor 340 filters the one or more blood flow signals to suppress (for example, remove or cancel out) the one or more human motion signals.

In block 1440, the processor 340 monitors the heart rate based on the one or more filtered blood flow signals.

Although FIG. 14 illustrates an example method 1400 for laser sensor system for direct heart rate signal measurement without interference from human body motion, various changes may be made to FIG. 14. For example, while shown as a series of steps, various steps in FIG. 14 could overlap, occur in parallel, occur in a different order, or occur any number of times. As a particular example, filtering the one or more human motion signals out from the one or more blood flow signals at block 1430 can include block 1432 of applying a sequential signal processing model, such as the SSP model 700 of FIG. 7. As another particular example, filtering the one or more human motion signals out from the one or more blood flow signals at block 1430 can include block 1434 of applying an optical speckle-based laser sensing algorithm, such as the process 1300 of FIG. 13.

Applying a sequential signal processing model can include: extracting, by a first long short-term memory (LSTM) cell and from the one or more blood flow signals, a first set of machine learning (ML) features configured to improve the estimation of the heart rate at a current time and a second set of ML features configured to improve a second estimation of the heart rate at a second time. Applying a sequential signal processing model can include extracting, by a second LSTM cell and from the one or more human motion signals and the first set of ML features, a third set of ML features configured to improve the second estimation of the heart rate. Applying a sequential signal processing model can include generating the estimation of the heart rate at the current time based on the one or more human motion signals and the first set of ML features. Applying a sequential signal processing model can further include: extracting, from the one or more blood flow signals detected at the second time, a fourth set of ML features configured to improve the second estimation of the heart rate and a fifth set of ML features configured to improve a third estimation of the heart rate based on the first set of ML features back fed into the first LSTM cell. Applying a sequential signal processing model can further include: extracting, from the one or more human motion signals detected at the second time and the fourth set of ML features, a sixth set of ML features configured to improve the third estimation of the heart rate based on the third set of ML features back fed into the second LSTM cell. Applying a sequential signal processing model can further include generating the second estimation of the heart rate at the second time based on the one or more human motion signals detected at the second time and the third set of ML features.

In some embodiments, when the optical speckle-based laser sensing algorithm is applied at block 1434, the spatially offset pair of laser transmitter and laser receiver includes a first laser transmitter that includes a polarizer and a first laser receiver that is a two-dimensional image sensor, and the spatially adjacent pair laser transmitter and laser receiver includes the first laser transmitter and a second laser receiver that is another two-dimensional image sensor that includes a polarized filter corresponding to the polarizer.

The above flowcharts illustrate example methods that can be implemented in accordance with the principles of the present disclosure and various changes could be made to the methods illustrated in the flowcharts herein. For example, while shown as a series of steps, various steps in each figure could overlap, occur in parallel, occur in a different order, or occur multiple times. In another example, steps may be omitted or replaced by other steps.

Although the figures illustrate different examples of user equipment, various changes may be made to the figures. For example, the user equipment can include any number of each component in any suitable arrangement. In general, the figures do not limit the scope of this disclosure to any particular configuration(s). Moreover, while figures illustrate operational environments in which various user equipment features disclosed in this patent document can be used, these features can be used in any other suitable system.

Although the present disclosure has been described with exemplary embodiments, various changes and modifications may be suggested to one skilled in the art. It is intended that the present disclosure encompass such changes and modifications as fall within the scope of the appended claims. None of the description in this application should be read as implying that any particular element, step, or function is an essential element that must be included in the claims scope. The scope of patented subject matter is defined by the claims.

What is claimed is:

1. A method comprising:
detecting one or more blood flow signals under human skin for estimation of a heart rate, using a spatially offset pair of laser transmitter and laser receiver;
detecting one or more human motion signals, using a spatially adjacent pair of laser transmitter and laser receiver;
reducing a motion interference signal within the one or more blood flow signals by filtering the one or more human motion signals out from the one or more blood flow signals; and
monitoring the heart rate based on the one or more filtered blood flow signals.

2. The method of claim 1, wherein filtering the one or more human motion signals out from the one or more blood flow signals comprises applying a sequential signal processing model including:
extracting, by a first long short-term memory (LSTM) cell and from the one or more blood flow signals, a first set of machine learning (ML) features configured to improve the estimation of the heart rate at a current time and a second set of ML features configured to improve a second estimation of the heart rate at a second time;
extracting, by a second LSTM cell and from the one or more human motion signals and the first set of ML features, a third set of ML features configured to improve the second estimation of the heart rate; and
generating the estimation of the heart rate at the current time based on the one or more human motion signals and the first set of ML features.

3. The method of claim 2, wherein applying the sequential signal processing model further comprises:
extracting, from the one or more blood flow signals detected at the second time, a fourth set of ML features configured to improve the second estimation of the heart rate and a fifth set of ML features configured to improve a third estimation of the heart rate based on the first set of ML features back fed into the first LSTM cell;
extracting, from the one or more human motion signals detected at the second time and the fourth set of ML features, a sixth set of ML features configured to improve the third estimation of the heart rate based on the third set of ML features back fed into the second LSTM cell; and
generating the second estimation of the heart rate at the second time based on the one or more human motion signals detected at the second time and the third set of ML features.

4. The method of claim 1, wherein filtering the one or more human motion signals out from the one or more blood flow signals comprises applying an optical speckle-based laser sensing algorithm; and
wherein the spatially offset pair of laser transmitter and laser receiver includes a first laser transmitter that includes a polarizer and a first laser receiver that is a two-dimensional image sensor; and
the spatially adjacent pair laser transmitter and laser receiver includes the first laser transmitter and a second laser receiver that is another two-dimensional image sensor that includes a polarized filter corresponding to the polarizer.

5. The method of claim 1, wherein within the spatially offset pair of laser transmitter and laser receiver:
laser receiver includes a cross-polarized filter.

6. The method of claim 1, wherein within the spatially adjacent pair laser transmitter and laser receiver:
the laser receiver includes a polarized filter.

7. The method of claim 1, wherein:
the spatially offset pair of laser transmitter and laser receiver includes a first laser transmitter that includes a polarizer and a first laser receiver; and
the spatially adjacent pair laser transmitter and laser receiver includes a second laser transmitter that includes a cross-polarizer and the first laser receiver.

8. An electronic device comprising:
a spatially offset pair of laser transmitter and laser receiver;
a spatially adjacent pair of laser transmitter and laser receiver; and
a processor operably connected to each laser transmitter and each laser receiver from among the spatially offset pair and the spatially adjacent pair, the processor configured to:
detect one or more blood flow signals under human skin for estimation of a heart rate, using a spatially offset pair of laser transmitter and laser receiver;
detect one or more human motion signals, using a spatially adjacent pair of laser transmitter and laser receiver;
reduce a motion interference signal within the one or more blood flow signals by filtering the one or more human motion signals out from the one or more blood flow signals; and
monitor the heart rate based on the one or more filtered blood flow signals.

9. The electronic device of claim 8, wherein to filter the one or more human motion signals out from the one or more blood flow signals, the processor is further configured to apply a sequential signal processing model including to:
extract, by a first long short-term memory (LSTM) cell and from the one or more blood flow signals, a first set of machine learning (ML) features configured to improve the estimation of the heart rate at a current time and a second set of ML features configured to improve a second estimation of the heart rate at a second time;
extract, by a second LSTM cell and from the one or more human motion signals and the first set of ML features, a third set of ML features configured to improve the second estimation of the heart rate; and
generate the estimation of the heart rate at the current time based on the one or more human motion signals and the first set of ML features.

10. The electronic device of claim 9, wherein to apply the sequential signal processing model, the processor is further configured to:
extract, from the one or more blood flow signals detected at the second time, a fourth set of ML features configured to improve the second estimation of the heart rate and a fifth set of ML features configured to improve a third estimation of the heart rate based on the first set of ML features back fed into the first LSTM cell;
extract, from the one or more human motion signals detected at the second time and the fourth set of ML features, a sixth set of ML features configured to improve the third estimation of the heart rate based on the third set of ML features back fed into the second LSTM cell; and
generate the second estimation of the heart rate at the second time based on the one or more human motion signals detected at the second time and the third set of ML features.

11. The electronic device of claim 8, wherein to filter the one or more human motion signals out from the one or more blood flow signals, the processor is further configured to apply an optical speckle-based laser sensing algorithm; and
wherein the spatially offset pair of laser transmitter and laser receiver includes a first laser transmitter that includes a polarizer and a first laser receiver that is a two-dimensional image sensor; and
the spatially adjacent pair laser transmitter and laser receiver includes the first laser transmitter and a second laser receiver that is another two-dimensional image sensor that includes a polarized filter corresponding to the polarizer.

12. The electronic device of claim 8, wherein within the spatially offset pair of laser transmitter and laser receiver:
laser receiver includes a cross-polarized filter.

13. The electronic device of claim 8, wherein within the spatially adjacent pair laser transmitter and laser receiver:
the laser receiver includes a polarized filter.

14. The electronic device of claim 8, wherein:
the spatially offset pair of laser transmitter and laser receiver includes a first laser transmitter that includes a polarizer and a first laser receiver; and
the spatially adjacent pair laser transmitter and laser receiver includes a second laser transmitter that includes a cross-polarizer and the first laser receiver.

15. A non-transitory computer readable medium embodying a computer program, the computer program comprising computer readable program code that when executed causes at least one processor to:
detect one or more blood flow signals under human skin for estimation of a heart rate, using a spatially offset pair of laser transmitter and laser receiver;
detect one or more human motion signals, using a spatially adjacent pair of laser transmitter and laser receiver;
reduce a motion interference signal within the one or more blood flow signals by filtering the one or more human motion signals out from the one or more blood flow signals; and
monitor the heart rate based on the one or more filtered blood flow signals.

16. The non-transitory computer readable medium of claim 15, wherein the program code that when executed causes the at least one processor to filter the one or more human motion signals out from the one or more blood flow signals further comprises program code that when executed causes the at least one processor to apply a sequential signal processing model, including to:
extract, by a first long short-term memory (LSTM) cell and from the one or more blood flow signals, a first set of machine learning (ML) features configured to improve the estimation of the heart rate at a current time and a second set of ML features configured to improve a second estimation of the heart rate at a second time;
extract, by a second LSTM cell and from the one or more human motion signals and the first set of ML features, a third set of ML features configured to improve the second estimation of the heart rate; and
generate the estimation of the heart rate at the current time based on the one or more human motion signals and the first set of ML features.

17. The non-transitory computer readable medium of claim 16, wherein the program code that when executed causes the at least one processor to apply the sequential signal processing model further comprises program code that when executed causes the at least one processor to:
extract, from the one or more blood flow signals detected at the second time, a fourth set of ML features configured to improve the second estimation of the heart rate and a fifth set of ML features configured to improve a third estimation of the heart rate based on the first set of ML features back fed into the first LSTM cell;
extract, from the one or more human motion signals detected at the second time and the fourth set of ML features, a sixth set of ML features configured to improve the third estimation of the heart rate based on the third set of ML features back fed into the second LSTM cell; and
generate the second estimation of the heart rate at the second time based on the one or more human motion signals detected at the second time and the third set of ML features.

18. The non-transitory computer readable medium of claim 15, wherein within the spatially offset pair of laser transmitter and laser receiver:
laser receiver includes a cross-polarized filter.

19. The non-transitory computer readable medium of claim 15, wherein within the spatially adjacent pair laser transmitter and laser receiver:
the laser receiver includes a polarized filter.

20. The non-transitory computer readable medium of claim 15, wherein the spatially offset pair of laser transmitter and laser receiver includes a first laser transmitter that includes a polarizer and a first laser receiver; and
the spatially adjacent pair laser transmitter and laser receiver includes a second laser transmitter that includes a cross-polarizer and the first laser receiver.
